(19) United States
(12) Patent Application Publication
(10) Pub. No.: US 2011/0014644 A1
(43) Pub. Date: Jan. 20, 2011

(54) METHODS FOR PREDICTING A CANCER PATIENT'S RESPONSE TO ANTIFOLATE CHEMOTHERAPY

(75) Inventors: Sarah L. Suchy, Pittsburgh, PA (US); Stacey L. Brower, Pittsburgh, PA (US); Paul R. Ervin, Pittsburgh, PA (US)

Correspondence Address: COOLEY LLP;ATTN: Patent Group, Suite 1100, 777 - 6th Street, NW, WASHINGTON, DC, 20001, US

(73) Assignee: Precision Therapeutics, Inc., Pittsburgh, PA (US)

(21) Appl. No.: 12/820,573

(22) Filed: Jun. 22, 2010

Related U.S. Application Data
(60) Provisional application No. 61/219,129, filed on Jun. 22, 2009.

(51) Int. Cl. C12Q 1/18

(52) U.S. Cl. 435/32

(57) ABSTRACT

The present invention provides methods for individualizing therapy for cancer treatment, and particularly for evaluating a patient's responsiveness to one or more antifolate therapeutic agents prior to treatment with such agents. Particularly, the invention provides an in vitro chemoresponse assay for predicting a patient's response to an antifolate agent, such as pemetrexed or methotrexate.

### CROSS REFERENCE TO RELATED APPLICATIONS

This application claims priority to Provisional Application No. 61/219,129, filed Jun. 22, 2009, which is hereby incorporated by reference in its entirety.

### FIELD OF THE INVENTION

The present invention relates to individualizing cancer treatment, and particularly to individualizing cancer treatment by evaluating a patient tumor specimen for its responsiveness to antifolate therapy prior to treatment.

### BACKGROUND

In an attempt to individualize cancer treatment, in vitro drug-response assay systems (chemoresponse assays) have been developed to predict the potential efficacy of chemotherapy agents for a given patient prior to their administration. Such systems, which include the MTT assay and the differential staining cytotoxicity (DiSC) assay, are not considered to produce reliable results for all chemotherapeutic agents. For example, the effectiveness of antifolate chemotherapeutic agents such as methotrexate, which target folic acid synthesis (a metabolite needed for de novo synthesis of thymidine and purine bases), can be difficult to evaluate in vitro. This is because the salvage of thymidine and purines will protect cells from the cytotoxic affects. See, e.g., Rots MG, Differential methotrexate resistance in childhood T-versus common/PreB-Acute Lymphoblastic Leukemia can be measured by an in situ thymidylate synthase inhibition, but not by the MTT assay, Blood 93(3): 1067-1074 (1999).

Pemetrexed (Alimta®) is a chemotherapeutic drug that is commonly used as a treatment for recurrent/advanced non-small cell lung carcinoma (NSCLC). Pemetrexed is an antifolate that operates by interfering with the activity of three folate-dependent enzymes—thymidylate synthase (TS), dihydrofolate reductase (DHFR), and glycinamide ribonucleotide formyltransferase (GARFT). Folic acid is a key component of nucleic acid synthesis. Although pemetrexed can produce a significant response is some patients, its overall population response is not impressive (9.1%). See, Hanna N., Randomized Phase III Trial of Pemetrexed Versus Docetaxel in Patients With Non-Small-Cell Lung Cancer Previously Treated With Chemotherapy, J. Clin. Oncol. 22: 1589-1597 (2004). Because of this low population response rate, assays for accurately discriminating responsive and non-responsive patients prior to treatment are needed to assist clinical decision making.

### SUMMARY OF THE INVENTION

The present invention provides methods for individualizing chemotherapy for cancer treatment, and particularly for evaluating a patient's responsiveness to one or more antifolate therapeutic agents prior to treatment with such agents. Particularly, the invention provides an in vitro chemoresponse assay for predicting a patient's response to an antifolate agent, such as pemetrexed or methotrexate.

The method generally comprises expanding malignant cells in culture from a patient's specimen (e.g., biopsy specimen), contacting the cultured cells with the antifolate therapeutic agent (e.g., pemetrexed or methotrexate), and evaluating or quantifying the response to the drug. In certain embodiments, monolayer(s) of malignant cells are cultured from explants prepared by mincing the tumor tissue, and the cells of the monolayer are suspended and plated for chemosensitivity testing. The result of the assay is a dose response curve, which may be evaluated using algorithms described herein, so as to quantitatively assess sensitivity to the antifolate therapeutic agent. The in vitro response to the drug as determined by the method of the invention is correlative with the patient's in vivo response upon receiving the antifolate during treatment (e.g., in the course of standardized or individualized therapeutic regimen).

In some embodiments, monolayer cultures are grown in the presence of growth medium that contains sufficient folic acid to support monolayer growth, and then treated with the anti-folate therapeutic agent in the presence of low folic acid medium (e.g., RPMI). In some embodiments, the growth medium is washed, rinsed, and/or diluted prior to treatment in low folic acid medium.

In some embodiments, the patient has lung cancer, such as non-small cell lung cancer (NSCLC), small cell lung cancer, or mesothelioma. As disclosed herein, studies with primary cultures of NSCLC specimens demonstrate a high degree of response heterogeneity to pemetrexed. When the response of 65 primary cultures of lung cancer were tested, 6.2% (4 of 65) of cultures responded to pemetrexed as a single agent, 9.2% (6 of 65) were determined to have an intermediate response, and 84.6% (55 of 65) were determined to be non-responsive. These results are consistent with the reported response rate for pemetrexed as a second-line treatment in the clinical setting (9.1% objective response).

These results suggest that pemetrexed efficacy, or other antifolate drug, can be evaluated in vitro using the cell-based chemoresponse assay disclosed herein, to determine which patients might benefit from this agent, and thereby avoiding unnecessary treatment in patients for which the drug is not efficacious.

### DESCRIPTION OF THE FIGURES

FIG. 1 shows the response of immortalized adenocarcinoma cell line A549 cultured in 5% BEGM media and then treated with pemetrexed in: 5% BEGM (Control) or 5% RPMI+EGF after undergoing a wash-before-treatment media change procedure.

FIG. 2 shows that 65 primary cultures of NSCLC specimens demonstrated a high degree of response heterogeneity to pemetrexed when tested at ten serially diluted concentrations between 2.24 nM and 586 μM. 6.2% (4 of 65) of cultures responded to pemetrexed as a single agent, 9.2% (6 of 65) were determined to have an intermediate response, and 84.6% (55 of 65) were determined to be non-responsive. These results are consistent with the reported response rate for pemetrexed as a second-line treatment in the clinical setting (9.1% objective response).

FIG. 3 shows the response of A549 cells cultured in 5% BEGM and switched into 5% RPMI+EGF media containing increasing concentrations of folic acid at the time of pemetrexed treatment.

### DETAILED DESCRIPTION OF THE INVENTION

The present invention provides methods for individualizing therapy for cancer treatment, and particularly, provides an in vitro chemoresponse assay for evaluating a patient's responsiveness to one or more antifolate therapeutic agents prior to treatment with such agents. The method generally comprises culturing and expanding the malignant cells from a patient's specimen (e.g., biopsy), contacting the cultured cells with an antifolate therapeutic agent (e.g., pemetrexed or methotrexate), and evaluating and/or quantifying the response to the drug. The in vitro response to the drug as determined by the method of the invention is correlative (e.g., predictive) of the patient's in vivo response upon receiving the antifolate during a therapeutic regimen.

The patient generally has a cancer for which an antifolate such as pemetrexed or methotrexate is a candidate treatment, for example, alone or in combination with other therapy. For example, the cancer may be selected from lung, mesothelioma, breast, ovarian, colorectal, endometrial, thyroid, nasopharynx, prostate, head and neck, liver, kidney, pancreas, bladder, and brain. In certain embodiments, the tumor is a solid tissue tumor and/or is epithelial in nature. For example, the cancer may be lung cancer, including NSCLC or small cell lung cancer, or in some embodiments, mesothelioma.

The present invention involves conducting chemoresponse testing with one or a panel of chemotherapeutic agents on cultured cells from the cancer patient, including with one or more antifolate chemotherapeutic agents. In certain embodiments, the chemoresponse method is as described in U.S. Pat. Nos. 5,728,541, 6,900,027, 6,887,680, 6,933,129, 6,416,967, 7,112,415, 7,314,731, and 7,501,260 (all of which are hereby incorporated by reference in their entireties). The chemoresponse method may further employ the variations described in US Published Patent Application Nos. 2007/0059821 and U.S. Pat. No. 7,642,048, both of which are hereby incorporated by reference in their entireties.

Briefly, in certain embodiments, cohesive multicellular particulates (explants) are prepared from a patient's tumor tissue sample (e.g., a biopsy sample) using mechanical fragmentation. This mechanical fragmentation of the explant may take place in a media substantially free of enzymes that are capable of digesting the explant. However, in some embodiments, some limited enzymatic treatment may be conducted, for example, to help reduce the size of the explants. Generally, the tissue sample is systematically minced using two sterile scalpels in a scissor-like motion, or mechanically equivalent manual or automated opposing incisor blades. This cross-cutting motion creates smooth cut edges on the resulting tissue multicellular particulates. The tumor particulates each measure from about 0.25 to about 1.5 mm³, for example, about 1 mm³.

After the tissue sample has been minced, the particles are plated in culture flasks (e.g., about 5 to 25 explants per flask). For example, about 9 explants may be plated per T-25 flask, or about 20 particulates may be plated per T-75 flask. For purposes of illustration, the explants may be evenly distributed across the bottom surface of the flask, followed by initial inversion for about 10-15 minutes. The flask may then be placed in a non-inverted position in a 37° C. CO₂ incubator for about 5-10 minutes. Flasks are checked regularly for growth and contamination. Over a period of a few days to a few weeks a cell monolayer will form. Further, it is believed (without any intention of being bound by the theory) that tumor cells grow out from the multicellular explant prior to stromal cells, such as fibroblasts and macrophages that may be initially present within the explants. Thus, by initially maintaining the tissue cells within the explant and removing the explant before the emergence of substantial numbers of stromal cells (e.g., at about 10 to about 50 percent confluency, or at about 15 to about 25 percent confluency), growth of the tumor cells (as opposed to substantial numbers of stromal cells) into a monolayer is facilitated. Further, in certain embodiments, the tumor sample or explants may be agitated to help release tumor cells from the tumor explant, and the tumor cells cultured to produce a monolayer. For example, the tumor specimen or explants may be agitated by placing the specimen or explants in a container, and shaking, rocking, or swirling the container, or striking the container against a hard surface.

The use of this procedure to form a cell culture monolayer helps maximize the growth of representative tumor cells from the tissue sample, and the resulting monolayer may comprise greater than about 60% (malignant) epithelial cells, or greater than about 70%, or greater than about 80%, or greater than about 90% (malignant) epithelial cells. The epithelial and/or malignant character of the cells may be confirmed using standard techniques. For example, see U.S. Pat. No. 7,642,048, which is hereby incorporated by reference in its entirety.

Prior to the chemotherapy assay, the growth of the cells may be monitored, and data from periodic counting may be used to determine growth rates which may or may not be considered parallel to growth rates of the same cells in vivo in the patient. Monolayer growth rate and/or cellular morphology and/or epithelial character may be monitored using, for example, a phase-contrast inverted microscope. Generally, the monolayers are monitored to ensure that the cells are actively growing at the time the cells are suspended for drug exposure. Thus, the monolayers will be non-confluent when the cells are suspended for chemoresponse testing.

Generally, the agents are tested against the cultured cells using plates such as microtiter plates. For the chemosensitivity assay, a reproducible number of cells is delivered to a plurality of wells on one or more plates, preferably with an even distribution of cells throughout the wells. For example, cell suspensions are generally formed from the monolayer cells before substantial phenotypic drift of the tumor cell population occurs. The cell suspensions may be, without limitation, about 4,000 to 12,000 cells/ml, or may be about 4,000 to 9,000 cells/ml, or about 7,000 to 9,000 cells/ml. The individual wells for chemoresponse testing are inoculated with the cell suspension, with each well or “segregated site” containing about 10² to 10⁴ cells. The cells are generally cultured in the segregated sites for about 4 to about 30 hours prior to contact with an agent.

Generally, the media used during the chemoresponse testing should contain, among other things, limited amounts of folic acid, in order to prevent the cells from escaping the cytotoxic effects of the antifolate therapeutic agent. In some embodiments, the treatment media is RPMI or similar media. For example, the media may contain the level of folic acid, purine nucleotide, and/or thymidine, as present in RPMI media. In certain embodiments, the media may contain less than about 2 μg/ml folic acid, such as about 1 μg/ml of folic acid (e.g., from about 0.2 or about 0.5 μg/ml to about 1.5 μg/ml). Such embodiments may be particularly helpful for testing lung specimens that are cultured in BEGM, which contains high amounts of folic acid.

In certain embodiments, monolayer cultures are grown in the presence of growth medium that contains sufficient folic acid to support monolayer growth (e.g., BEGM), and then treated with the anti-folate therapeutic agent in the presence of low folic acid medium (e.g., RPMI). The growth medium may be washed, rinsed, and/or diluted from the cells prior to treatment in low folic acid medium. Such a “wash-before-treatment” (WBT) procedure allows the malignant cells to be cultured in virtually any type of media, and then switched to low folic acid media, such as RPMI, for drug treatment. Thus, the cells may remain in a media optimal for cell growth, while conducting sensitivity testing in a media optimal for quantifying the drug response.

In certain embodiments, this WBT procedure is performed by an automated liquid handler that accurately controls pipette aspiration/speed, to avoid cell dissociation that may occur with more uncontrolled or harsh manual pipetting. Cell dissociation can lead to erratic cell counts.

Because simple aspiration will not remove all growth media from a well (thereby leaving sufficient folic acid behind to potentially affect dose response curves), after removal of growth media and prior to the addition of low folic acid treatment media, cultured cells are preferably washed with a solution containing no folic acid (e.g., a buffered solution such as PBS). In some embodiments, any remaining growth media after the wash is diluted by adding an excess (e.g., about 1.5 to about 2-fold excess) of low folic acid media (i.e. 70 μL), and the excess media (i.e. about 30 μL) is removed leaving the desired treatment volume (e.g., 40 μL).

Each test well is then contacted with at least one pharmaceutical agent, or a sequence of agents.

A series of test wells is contacted with a serial dilution of an antifolate therapeutic agent, such as pemetrexed or methotrexate. In some embodiments, the antifolate therapeutic agent is pemetrexed. Pemetrexed is significantly less toxic than agents such as docetaxel which might be used with similar recurrent patients. Pemetrexed is an antifolate that operates by interfering with the activity of three folate-dependent enzymes—thymidylate synthase (TS), dihydrofolate reductase (DHFR), and glycinamide ribonucleotide formyltransferase (GARFT). Folic acid is a key component of nucleic acid synthesis. Folic acid is converted to tetrahydrofolate by the actions of DHFR, and then to thymidine by TS. Pemetrexed interferes with the enzymes in this process in a competitive manner. Because the activity is competitive, more folic acid results in less inhibition of tumor growth by pemetrexed. For this reason, it is important that patients receiving pemetrexed not take vitamins or eat a diet that is high in folic acid regularly, but rather, be treated with vitamin B12 at 9 week intervals and with low level folate (350-1000 μg orally) daily to control toxicity.

In addition to at least one antifolate chemotherapeutic agent, the panel of chemotherapeutic agents may comprise at least one agent selected from a platinum-based drug, a taxane, a nitrogen mustard, a kinase inhibitor, an EGFR inhibitor (e.g., tyrosine kinase inhibitor or antibody targeting the extracellular domain), a pyrimidine analog, a podophyllotoxin, an anthracycline, a monoclonal antibody, and a topoisomerase I inhibitor. For example, the panel may comprise 1, 2, 3, 4, or 5 agents selected from bevacizumab, capecitabine, carboplatin, cecetuximab, cisplatin, cyclophosphamide, docetaxel, doxorubicin, epirubicin, etoposide, 5-fluorouracil, gemcitabine, irinotecan, oxaliplatin, paclitaxel, panitumumab, tamoxifen, topotecan, and trastuzumab, in addition to other potential agents for treatment. In certain embodiments, the chemoresponse testing includes one or more combination treatments, such combination treatments including one or more agents described above. Generally, each agent in the panel is tested in the chemoresponse assay at a plurality of concentrations representing a range of expected extracellular fluid concentrations upon therapy.

For example, pemetrexed may be tested at concentrations of about 0.03 ng/ml to about 20 mg/ml, or from about 0.1 ng/ml to about 15 mg/ml, or from about 1.0 ng/ml or 0.03 mg/ml to about 10 mg/ml. Pemetrexed may be tested at concentrations of about 1.0 nM to about 1.0 mM, or from about 20 nM to about 600 μM, or from about 2.24 nM to about 586 μM. Such concentrations of pemetrexed demonstrated heterogeneous responses across patient samples.

The efficacy of each agent in the panel is determined against the patient's cultured cells, by determining the viability of the cells (e.g., number of viable cells). For example, at predetermined intervals before, simultaneously with, or beginning immediately after, contact with each agent or combination, an automated cell imaging system may take images of the cells using one or more of visible light, UV light and fluorescent light. Alternatively, the cells may be imaged after about 25 to about 200 hours of contact with each treatment (e.g., about 3 days, or about 72 hours). The cells may be imaged once or multiple times, prior to or during contact with each treatment. Of course, any method for determining the viability of the cells may be used to assess the efficacy of each treatment in vitro.

While any grading system may be employed, in certain embodiments the grading system may employ from 2 to 10 response levels, e.g., about 3, 4, or 5 response levels. For example, when using three response grades, the three grades may correspond to a responsive grade, an intermediate responsive grade, and a non-responsive grade. In certain embodiments, the patient's cells show a heterogeneous response across the panel of agents, making the selection of an agent particularly crucial for the patient's treatment.

The output of the assay is a series of dose-response curves for tumor cell survivals under the pressure of a single or combination of drugs, with multiple dose settings each (e.g., ten dose settings). To better quantify the assay results, the invention employs in some embodiments a scoring algorithm accommodating a dose-response curve. Specifically, the chemoresponse data are applied to an algorithm to quantify the chemoresponse assay results by determining an adjusted area under curve (aAUC). The aAUC takes into account changes in cytotoxicity between dose points along a dose-response curve, and assigns weights relative to the degree of changes in cytotoxicity between dose points. For example, changes in cytotoxicity between dose points along a dose-response curve may be quantified by a local slope, and the local slopes weighted along the dose-response curve to emphasize cytotoxic responses.

For example, aAUC may be calculated as follows.

Step 1: Calculate Cytotoxity Index (CI) for each dose, where CI=Mean_{drug}/Mean_{control}.

Step 2: Calculate local slope (S_{d}) at each dose point, for example, as S_{d}=(CI_{d}−CI_{d-1})/Unit of Dose, or S_{d}=(CI_{d-1}−CI_{d})/Unit of Dose.

Step 3: Calculate a slope weight at each dose point, e.g., W_{d}=1−S_{d}.

Step 4: Compute aAUC, where aAUC=Σ W_{d} CI_{d}, and where, d=each dose, e.g., 1, 2, . . . , 10. Equation 4 is the summary metric of a dose response curve and may used for subsequent regression over reference outcomes.

Usually, the dose-response curves vary dramatically around middle doses, not in lower or higher dose ranges. Thus, the algorithm in some embodiments need only determine the aAUC for a middle dose range, such as for example (where from 8 to 12 doses are experimentally determined, e.g., 10 doses), the middle 4, 5, 6, or 8 doses are used to calculate aAUC. In this manner, a truncated dose-response curve might be more informative in outcome prediction by eliminating background noise.

The numerical aAUC value (e.g., test value) may then be evaluated for its effect on the patient's cells, and compared to the same metric for other drugs on the patient's cells. For example, a plurality of drugs may be tested, and aAUC determined as above for each, to determine whether the patient's cells have a sensitive response, intermediate response, or resistant response to each drug. Further, the measures may be compared to determine the most effective drug.

In some embodiments, each drug is designated as, for example, sensitive, or resistant, or intermediate, by comparing the aAUC test value to one or more cut-off values for the particular drug (e.g., representing sensitive, resistant, and/or intermediate aAUC scores). The cut-off values for any particular drug may be set or determined in a variety of ways, for example, by determining the distribution of a clinical outcome within a range of corresponding aAUC reference scores. That is, a number of patient tumor specimens are tested for chemosensitivity/resistance (as described herein) to a particular drug prior to treatment, and aAUC quantified for each specimen. Then after clinical treatment with that drug, aAUC values that correspond to a clinical response (e.g., sensitive) and the absence of significant clinical response (e.g., resistant) are determined. Cut-off values may alternatively be determined from population response rates. For example, where a patient population is known to have a response rate of 30% for the tested drug, the cut-off values may be determined by assigning the top 30% of aAUC scores for that drug as sensitive. Further still, cut-off values may be determined by statistical measures, such as mean or median scores.

In some embodiments, the aAUC cut-off value for a sensitive designation with pemetrexed is about 5.3 or less (e.g., as calculated herein). A cut-off value for a resistant designation with pemetrexed is about 6.5 or more (e.g., as calculated herein). An aAUC of between about 5.3 and about 6.5 is considered an intermediate response. In other embodiments, the aAUC scores may be a continuous scale.

### EXAMPLES

### Methods

The ChemoFx™ live cell chemoresponse assay was performed on 65 patient specimens and the cell line A549. The cells were treated with a 10-dose range of pemetrexed for 72 hours before DAPI-nuclear staining and counting. AUC (Area Under Curve) values were calculated and additional statistical analysis was performed on the resulting dose-response curves.

Folic Acid supplementation experiments were conducted. Pure folic acid was purchased from Sigma Aldrich, filtered, and prepared in a 100× concentration using RPMI media. The concentrate was added to RPMI media so that the final concentration of folic acid ranged between 1.5 mg/L and 500 mg/L. The cell line A549 was plated and treated according to the ChemoFx™ assay in the RPMI media containing increasing concentrations of folic acid. A control was also plated and treated at the same time.

The area under the dose-response curve (AUC) represents the survival fraction of cells in the presence of drug at each of the 10 increasing dose concentrations. There can be certain circumstances when dose-response curves of differing shapes share similar AUCs. Therefore, as a better reflection of sensitivity to drug treatment, an adjusted area under the curve (aAUC) was calculated according to the following formulas. First, local slope (Sd) at each dose point was calculated based on Sd=(CId-1−CId)/unit of dose. Second, slope weight (Wd) at each dose point was calculated based on Wd=1−Sd. Finally, aAUC was computed based on aAUC=Σ Wd CId, where d=1 . . . 10. Mi Z, et al. (2008) Anticancer Res 28: 1733-1740.

Adjusted areas under the curve (aAUC) were calculated for each dose-response curve. The assay results were classified as responsive (R; assay score≦5.34648.0), intermediate responsive (IR; assay score 5.3465-6.5372) or non-responsive (NR; assay score≧6.5373).

TABLE 1
Exemplary aAUC calculation

| Dose # | TARA1725 | | | | | | | |
|---|---|---|---|---|---|---|---|---|
| Dose | Rep1 | Rep2 | Rep3 | Xi | Xᵢ₋₁—Xᵢ | 1-(Xᵢ₋₁—Xᵢ) | [1-(Xᵢ₋₁—Xᵢ)]*Xᵢ | |
| 0 | 1.428 | 0.947 | 0.625 | 1.000 | | | | |
| 1 | 0.316 | 0.386 | 0.445 | 0.382 | 0.618 | 0.382 | 0.146 | |
| 2 | 0.273 | 0.282 | 0.444 | 0.333 | 0.049 | 0.951 | 0.316 | |
| 3 | 0.221 | 0.315 | 0.293 | 0.276 | 0.056 | 0.944 | 0.261 | |
| 4 | 0.252 | 0.302 | 0.344 | 0.299 | −0.023 | 1.023 | 0.306 | |
| 5 | 0.242 | 0.257 | 0.200 | 0.233 | 0.066 | 0.934 | 0.218 | |
| 6 | 0.335 | 0.201 | 0.264 | 0.267 | −0.033 | 1.033 | 0.275 | |
| 7 | 0.247 | 0.264 | 0.204 | 0.238 | 0.028 | 0.972 | 0.232 | |
| 8 | 0.264 | 0.132 | 0.147 | 0.181 | 0.057 | 0.943 | 0.171 | |
| 9 | 0.226 | 0.135 | 0.123 | 0.162 | 0.019 | 0.981 | 0.159 | |
| 10 | 0.108 | 0.114 | 0.084 | 0.102 | 0.059 | 0.941 | 0.096 | |
| | | | | | | | aAUC: | 2.179 |

These cut-off values are consistent with the reported clinical response rates for pemetrexed of about 9.1%.

In order to determine whether or not removal of excess folic acid from growth media affected the outcome of the chemoresponse assay, a Wash Before Treatment (WBT) step was included. Briefly, A549 cells were plated in 5% BEGM growth media and were cultured to the desired confluence. The growth media was then aspirated and the cells washed with 70 μL of PBS. After removal of the PBS, 70 μL of 5% RPMI+EGF (EGF concentration of 10 ng/mL) treatment media was added to the each treatment well. Following the addition of the treatment media, 30 μL of the treatment media was removed leaving a total volume of 40 μL of treatment media per well. Pemetrexed was then prepared using serial dilutions in the treatment media and was applied to the treatment wells. All aspirations and additions of media and wash solutions was performed by an automated liquid handler to avoid harsh pipetting that might dissociate/disrupt cells.

### Results

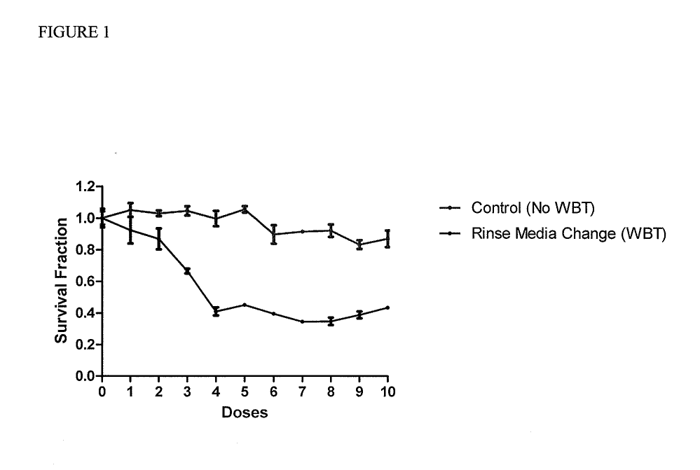

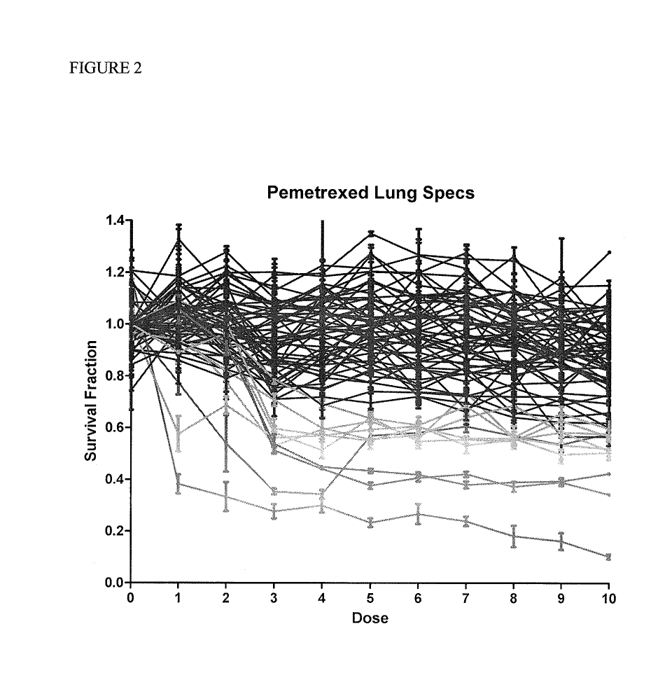

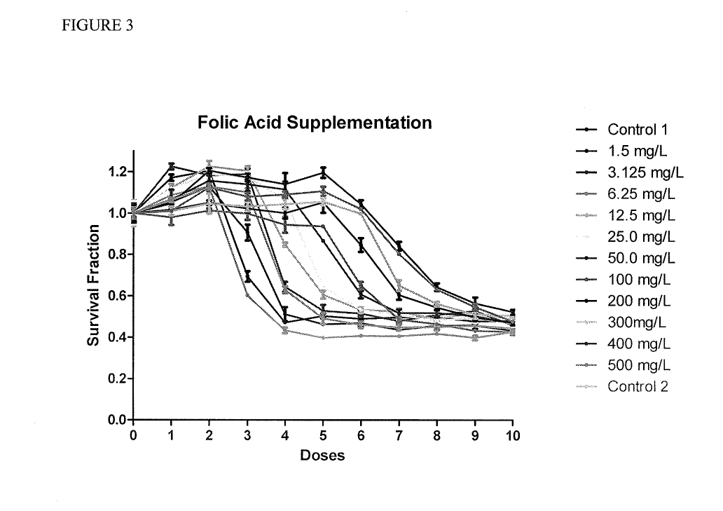

The immortalized lung adenocarcinoma cell line A549 was found to be a good positive control for sensitivity to pemetrexed treatment. FIG. 1. Studies with primary cultures of NSCLC specimens demonstrate a high degree of response heterogeneity when tested at ten serially diluted concentrations between 2.24 nM and 586 μM. FIG. 2. The observed activity of the drug could be reversed with folic acid treatment of the cell line. FIG. 3.

The addition of the WBT protocol to the chemosensitivity assay significantly improved the in vitro response to pemetrexed. FIG. 1. Without WBT, a typical ChemoFx™ response curve was not obtained for A549 cells.

When the response characteristics of 65 primary cultures of lung cancer were tested, it was demonstrated that 6.2% of patients responded to pemetrexed as a single agent, 9.2% had intermediate response and 84.6% were non-responsive. These results agree with the reported response rate for pemetrexed as a second-line treatment in the clinical setting (9.1% objective response). These results suggest that pemetrexed efficacy can be tested in vitro using the cell-based chemoresponse assay disclosed herein, to thereby determine which patients might benefit from this agent, thereby reducing unnecessary treatment in patients for which the drug is not efficacious.

All references cited herein, including all patent and non-patent literature, are hereby incorporated by reference for all purposes.

1. A method for predicting the efficacy of an antifolate therapeutic agent for a cancer patient, comprising:
culturing malignant cells from a tumor specimen from the patient;
contacting the cultured cells with the antifolate therapeutic agent; and
evaluating the response of the malignant cells to the drug.

2. The method of claim 1, wherein the cancer is selected from lung, mesothelioma, breast, ovarian, colorectal, endometrial, thyroid, nasopharynx, prostate, head and neck, liver, kidney, pancreas, bladder, and brain.

3. The method of claim 1, wherein the cancer is lung cancer.

4. The method of claim 3, wherein the lung cancer is NSCLC or mesothelioma.

5. The method of claim 1, wherein the antifolate therapeutic agent is pemetrexed.

6. The method of claim 1, wherein the malignant cells are cultured from a plurality of tumor explants in a monolayer culture in growth media.

7. The method of claim 6, wherein the tumor explants are prepared by mechanical fragmentation of the patient's tumor specimen.

8. The method of claim 7, wherein the tumor specimen is minced.

9. The method of claim 8, wherein the explants each measure from about 0.25 to about 1.5 mm³.

10. The method of claim 6, where the monolayer cells are suspended in growth media, and the cells plated for chemosensitivity testing in treatment media.

11. The method of claim 10, wherein the growth media is removed prior to adding the treatment media.

12. The method of claim 10, wherein the treatment media is low folic acid media.

13. The method of claim 12, wherein the low folic acid media is RPMI.

14. The method of claim 10, wherein the cells are washed between the removal of growth media and the addition of treatment media.

15. The method of claim 14, wherein the cells are washed with PBS.

16. The method of claim 10, wherein dilutions of pemetrexed are added across a plurality of wells within the range of about 0.03 ng/ml to about 20 mg/ml, or about 1.0 nM to about 1.0 mM.

17. The method of claim 1, wherein the drug is contacted with the cells for about 3 days, and then cell viability quantified.

18. The method of claim 17, wherein cell viability is quantified by visible light, UV light, or fluorescent light.

19. The method of claim 17, wherein cells are stained with DAPI.

20. The method of claim 1, wherein the response of the drug to the chemotherapeutic agent is evaluated by preparing a dose response curve, and determining an adjusted AUC.

21. The method of claim 20, wherein the aAUC assigns weights relative to the degree of change in cytotoxicity between dose points.

22. The method of claim 21, wherein changes in cytotoxicity between dose points are quantified by a local slope, and the local slopes weighted along the dose-response curve to emphasize cytotoxic responses.

23. The method of claim 22, wherein aAUC is calculated by:
calculating a Cytotoxity Index (CI) for each dose;
calculating a local slope (S_{d}) at each dose point;
calculating a slope weight at each dose point, by W_{d=1}−S_{d}; and
calculating aAUC, where aAUC=Σ W_{d} CI_{d}, and where d represents each dose in a range.

24. The method of claim 23, wherein aAUC is calculated for a truncated dose response curve.

25. The method of claim 23, further comprising, assigning the tumor sample as being sensitive, resistant, or intermediate sensitive to the chemotherapeutic agent.
